(19) United States
(12) Patent Application Publication
(10) Pub. No.: US 2018/0366219 A1
(43) Pub. Date: Dec. 20, 2018

(54) Hospital Information System

(71) Applicant: Canon Medical Systems Corporation, Otawara-shi (JP)

(72) Inventors: Masafumi Abe, Hirakata (JP); Tomoya Takefuji, Utsunomiya (JP)

(73) Assignee: Canon Medical Systems Corporation, Otawara-shi (JP)

(21) Appl. No.: 15/932,188

(22) Filed: Feb. 16, 2018

(30) Foreign Application Priority Data
Feb. 16, 2017 (JP) 2017-026872

(51) Int. Cl. G16H 40/20; G16H 10/60; G06F 17/30; G06Q 10/06

(52) U.S. Cl. CPC G16H 40/20; G06Q 10/06311; G06F 17/30312; G16H 10/60

(57) ABSTRACT

A hospital information system includes processing circuitry. The processing circuitry is configured to specify a work of each health professional by using determination of an event that relates to medical care to a subject as a trigger, the work relating to the event. The processing circuitry is configured to output display information representing the specified work of each health professional so that the health professionals are identifiable with respect to each work in the display information.

### CROSS-REFERENCE TO RELATED APPLICATIONS

This application is based upon and claims the benefit of priority from Japanese Patent Application No. 2017-026872, filed on Feb. 16, 2017; the entire contents of which are incorporated herein by reference.

### FIELD

Embodiments descried herein relate generally to a hospital information system.

### BACKGROUND

Conventionally, clinical pathways each defining a standard schedule of clinical practice and nursing care for each disease and each examination have been used in hospital information systems (HIS). For example, a hospital information system defines a clinical pathway according to the name of disease or injury at hospital admission and, by selecting the clinical pathway according to the name of disease or injury, the content of clinical practice and nursing care is developed into clinical instructions (orders).

### BRIEF DESCRIPTION OF THE DRAWINGS

FIG. 1 is a diagram for explaining an overview of a hospital information system according to a first embodiment;

FIG. 2 is a diagram illustrating an exemplary configuration of a hospital information system according to the first embodiment;

FIG. 3 is a diagram illustrating exemplary definition information according to the first embodiment;

FIG. 4 is a diagram illustrating exemplary registration information according to the first embodiment;

FIG. 5 is a diagram for explaining an exemplary task/work information generation process that is performed by a generating function according to the first embodiment;

FIG. 6 is a diagram for explaining an exemplary update process that is performed by an updating function according to the first embodiment;

FIG. 7 is a diagram illustrating an overview of output of task/work information that is performed by an outputting function according to the first embodiment;

FIG. 8 is a diagram representing exemplary display information that is output by the outputting function according to the first embodiment;

FIG. 9 is a diagram representing exemplary display information that is output by the outputting function according to the first embodiment;

FIG. 10 is a diagram for explaining a determination process that is performed by an updating function according to the first embodiment;

FIG. 11 is a diagram for explaining the determination process that is performed by the updating function according to the first embodiment;

FIG. 12 is a diagram for explaining the determination process that is performed by the updating function according to the first embodiment;

FIG. 13 is a diagram for explaining the determination process that is performed by the updating function according to the first embodiment;

FIG. 14 is a diagram for explaining the determination process that is performed by the updating function according to the first embodiment; and

FIG. 15 is a flowchart illustrating a process procedure that is performed by the hospital information system according to the first embodiment.

### DETAILED DESCRIPTION

A hospital information system according to an embodiment includes processing circuitry. The processing circuitry is configured to specify a work of each health professional by using determination of an event that relates to medical care to a subject as a trigger, the work relating to the event. The processing circuitry is configured to output display information representing the specified work of each health professional so that the health professionals are identifiable with respect to each work in the display information.

A hospital information system according to an embodiment includes processing circuitry. The processing circuitry is configured to specify a work of each health professional by using determination of an event that relates to medical care to a subject as a trigger, the work relating to the event. The processing circuitry is configured to output display information representing the specified work of each health professional.

With reference to the accompanying drawings, embodiments of the hospital information system and the medical information processing program according to the present application will be described in detail. The following embodiments do not limit the hospital information system and the medical information program according to the present application.

### First Embodiment

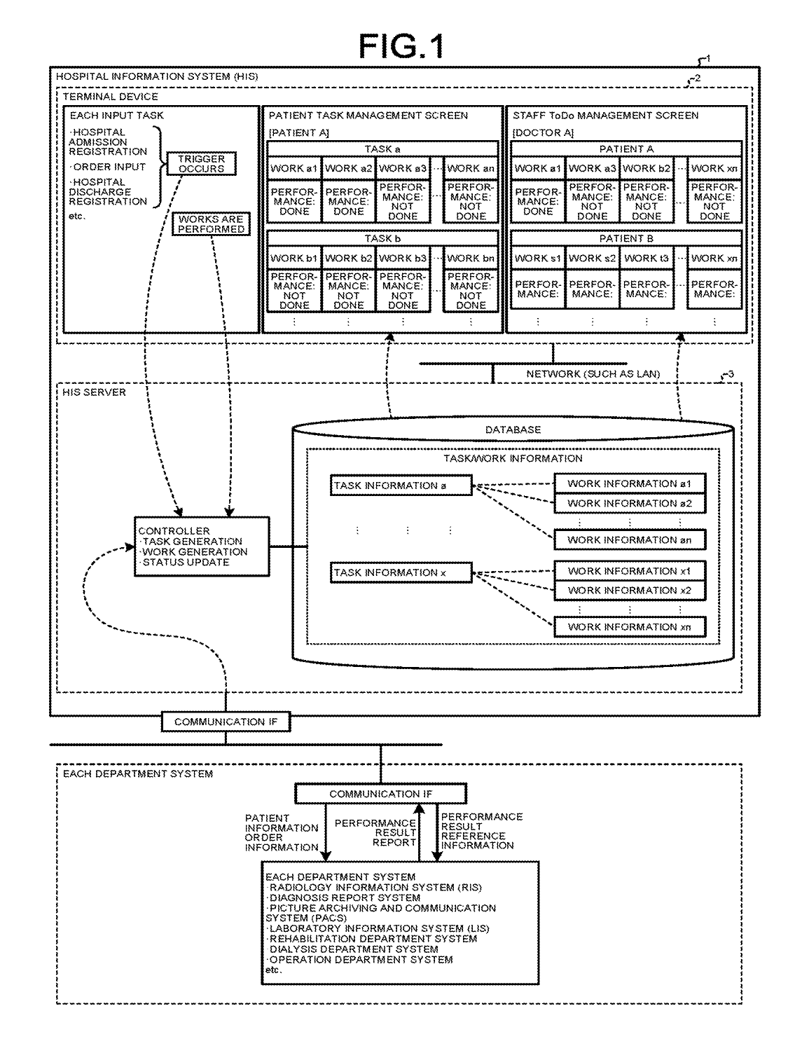

First of all, an overview of a hospital information system according to the first embodiment will be described. FIG. 1 is a diagram for explaining an overview of the information hospital system according to the first embodiment. As illustrated in FIG. 1, a hospital information system 1 according to the first embodiment includes a terminal device 2 and a HIS server 3 and is connected to each department system via a communication interface (IF). The terminal device 2 and the HIS server 3 are able to communicate with each other via, for example, an internal local area network (LAN) that is set in a hospital.

The department systems include various systems, such as a radiology information system (RIS), a diagnosis report system, a picture archiving and communication system (PACS), a laboratory information system (LIS), a rehabilitation department system, a dialysis department system, and an operation department system. The hospital information system 1 is connected to each of the department systems and transmits and receives various types of information to and from each of the department systems. For example, the hospital information system 1 transmits patient information and order information to each of the department systems and receives a report of the result of performance of each of the department systems. In response to a request from each of the department systems, the hospital information system 1 transmits the result of performance and reference information.

The hospital information system 1 according to the embodiment enables improvement of medical task efficiency by managing, in addition to medical tasks relating to clinical practice and nursing care, medical tasks other than clinical practice and nursing care (for example, creating various types of clinical documents, checking examination results and ordinary routine tasks) in the system. Specifically, an event that is determined with respect to a subject who visits a hospital triggers the hospital information system 1 to register and manage medical tasks relating to the event in the system and thus the hospital information system 1 improves medical task efficiency. The hospital information system 1 manages, in association with the medical task, the work of each health professional that is performed before completion of the medical task. In other words, the hospital information system 1 manages each of works of all the health professionals that relate to one medical task until the medical task completes, thereby improving medical task efficiency. The above-described medical tasks may be referred to as tasks below.

For example, as illustrated in FIG. 1, when each input task of, for example, hospital admission registration, order input, or hospital discharge registration is performed on the terminal device 2, a controller of the HIS server 3 is triggered by the performed input task to generate a task and works and register the task and the works in a database. For example, the controller generates task/work information in which task information a that relates to the input task serving as the trigger is associated with sets of work information al to an for performing the task of the task information a and registers the task/work information in the database.

The health professionals who perform the works include not only a doctor and a nurse but also a healthcare professional and clerical staff. In other words, the controller manages detailed works that are performed to perform one task in a medical institution, such as a hospital. Note that task/work information that is generated according to the trigger is not limited to one set of information. In other words, the controller is able to generate multiple sets of task/work information according to a single trigger.

On generating task/work information as described above, the controller of the HIS server 3 implements control to output the task/work information registered in the database to the terminal device 2 operated by the health professional. For example, as illustrated in FIG. 1, the controller implements control to display a task management screen for each subject (for example, a patient A) on the display of the terminal device 2. For example, the controller implements control to display the task a including the works al to an and a task b including works b1 to bn on the task management screen for the patient A. The controller further implements control to display a ToDo management screen for a staff (a doctor A) on the display of the terminal device 2. For example, the controller implements control to display a list of the patient A including a work al, a work a3, a work b2 and a work Xn and a list of a patient B including a work s1, a work s2, a work t3 and a work Xn on the ToDo management screen for the doctor A.

As described above, the hospital information system 1 segments each task in the medical institution into works of the respective health professionals including a healthcare professional and a clerical staff and manages the works and, by representing the content of work of each health professional to each health professional, enables each work to be performed speedily and thus improves medical task efficiency.

For example, application of the above-described clinical pathway enables the hospital information system to realize a system in which selection of the clinical pathway causes a medical task, including medical practice and nursing care, defined by the selected clinical pathway to be transmitted as order information to each department system. As descried above, with a clinical pathway, however, a medical task, including clinical practice and nursing care, performed by a doctor and a nurse is developed to order information. In other words, with a clinical pathway, it is possible to manage medical tasks including clinical practice and nursing care in the system; however, it is not possible to manage medical tasks other than clinical practice and nursing care (for example, creating various types of clinical documents and ordinary routine tasks) in the system.

In this case, for example, clinical documents (such as a hospitalization clinical plan document and a consent form) that have to be created in association with hospital admission and a clinical instruction are created individually by each task manager by hand or by using a clinical document system. Requests to, for example, fill in a document and for signature are made verbally or by internal e-mails. For this reason, tasks are sometimes not performed speedily due to delay in communications between staffs or miscommunication. On the other hand, the hospital information system 1 according to the first embodiment is able to segment a task into works, manage the works and represent information about the works to each health professional and thus enables each work to be performed speedily.

The controller of the HIS server 3 determines whether a work is performed on the basis of an event representing that the work is performed and updates the status of the task/work information. For example, as illustrated in FIG. 1, the controller implements control to display works that are performed as “performed” on the task management screen for each patient and the staff ToDo management screen. This enables a health professional to know the progress of each task and do the work to be performed by the health professional. Even if performance of a task delays, as it is possible to see which work delays, it is possible to prompt the responsible health professional to perform the delayed work and thus further improve the medical task efficiency.

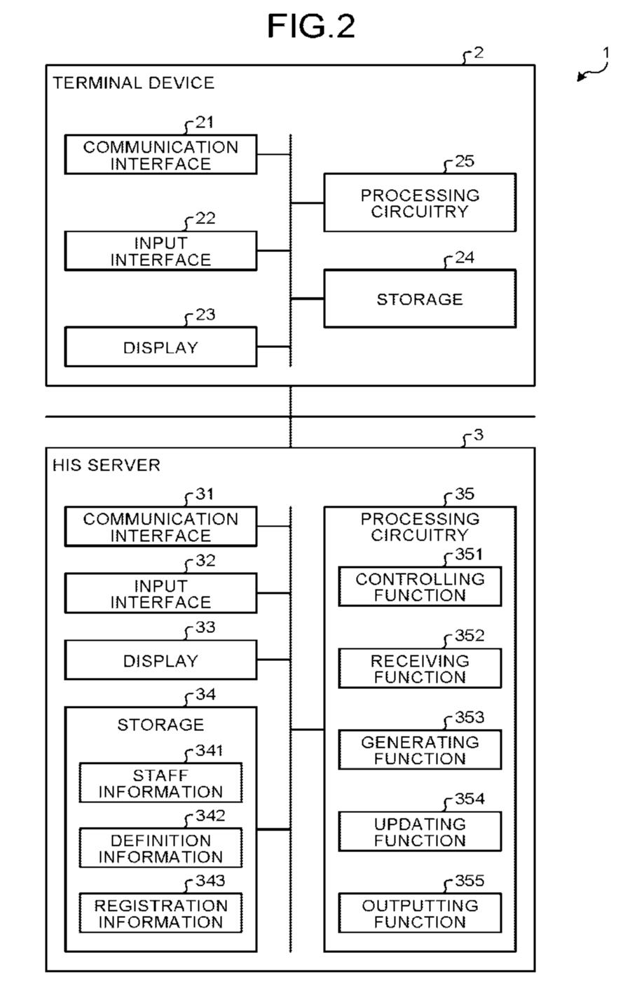

As described above, the hospital information system 1 according to the first embodiment improves the medical task efficiently by managing each of the works of all the health professionals that relate to one medical task until the medical task completes. Details of the hospital information system 1 will be described below. FIG. 2 is a diagram illustrating an exemplary configuration of the hospital information system 1 according to the first embodiment. As illustrated in FIG. 2, the hospital information system 1 according to the first embodiment includes the terminal device 2 and the HIS server 3 that are connected to each other via a network. FIG. 2 represents the single terminal device 2; however, practically, the multiple terminal devices 2 are connected to the HIS server 3 via the network. FIG. 2 does not represent various devices that relate to each department system; however, practically, the hospital information system 1 is directly or indirectly connected to, for example, a server device contained in each of the department systems such that the hospital information system 1 and the server device are able to communicate with each other. It is also possible to apply a clinical document system that supports creation of clinical documents to the hospital information system 1. In such a case, an operator, for example, operates the terminal device 2 to start the clinical document system to enable creation of a desired clinical document.

As illustrated in FIG. 2, the terminal device 2 includes a communication interface 21, an input interface 22, a display 23, storage 24, and processing circuitry 25. The communication interface 21 is connected to the processing circuitry 25 and controls transmission and communication of various types of data to and with the HIS server 3 to which the terminal device 2 is connected via the network. For example, the communication interface 21 is realized by a network card, a network adapter, or a network interface controller (NIC).

The input interface 22 is connected to the processing circuitry 25, converts an input operation that is received from an operator (health professional) into electric signals and outputs the electric signals to the processing circuitry 25. For example, the input interface 22 includes a switch button, a mouse, a keyboard and a touch panel. The input interface 22 receives an operation serving as a trigger in the hospital information system 1. For example, the input interface 22 receives an operation of inputting an event (for example, hospital admission registration or order input) that relates to medicine to a subject. The input interface 22 receives various input operations to perform works.

The display 23 is connected to the processing circuitry 25 and displays various types of information and various types of image data that are output from the processing circuitry 25. For example, the display 23 is realized by a liquid crystal monitor, a cathode ray tube (CRT) monitor, or a touch panel. The display 23 displays display information that is generated by the HIS server 3. For example, the display 23 displays task/work information that is generated by the HIS server 3.

The storage 24 is connected to the processing circuitry 25 and stores various types of data. For example, the storage 24 is realized by a semiconductor memory device, such as a random access memory (RAM) or a flash memory, a hard disk or an optical disk. In the embodiment, the storage 24 stores the task/work information that is received from the HIS server 3.

The processing circuitry 25 controls each component of the terminal device 2 according to an input operation that is received from the operator via the input interface 22. For example, the processing circuitry 25 is realized by a processor. In the embodiment, the processing circuitry 25 causes the communication interface 21 to transmit information corresponding to the input operation that is received via the input interface 22 to the HIS server 3. The processing circuitry 25 further causes the storage 24 to store the task/work information that is input from the communication interface 21. The processing circuitry 25 further reads the task/work information, etc., from the storage 24 and causes the display 23 to display the task/work information.

For example, the terminal device 2 is arranged in various specialties and departments in the hospital and is operated by medical specialists, such as doctors, nurses, healthcare professionals, and clerical staffs. Each health professional operates the input interface 22 of the terminal device 2 and input a staff ID of the health professional and thus is able to access the hospital information system 1. Each health professional performs an input operation serving as a trigger, an input operation to perform a work, and an input operation to browse task/work information. The terminal device 2 transmits information corresponding to an input operation that is executed by each health professional to the HIS server 3.

As illustrated in FIG. 2, the HIS server 3 includes a communication interface 31, an input interface 32, a display 33, storage 34 and processing circuitry 35 and executes a process corresponding to the information that is received from the terminal device 2. The communication interface 31 is connected to the processing circuitry 35 and controls transmission and communication of various types of data to and with the terminal device 2 to which the HIS server 3 is connected via the network. For example, the communication interface 31 is realized by a network card, a network adapter, or a network interface controller (NIC).

The input interface 32 is connected to the processing circuitry 35, converts an input operation that is received from the operator into electric signals and outputs the electric signals to the processing circuitry 35. For example, the input interface 32 includes a switch button, a mouse, a keyboard and a touch panel.

The display 33 is connected to the processing circuitry 35 and displays various types of information that are output from the processing circuitry 35. For example, the display 33 is realized by a liquid crystal monitor, a cathode ray tube (CRT) monitor, or a touch panel.

The storage 34 is connected to the processing circuitry 35 and stores various types of data. For example, the storage 34 is realized by a semiconductor memory device, such as a random access memory (RAM) or a flash memory, a hard disk or an optical disk. In the embodiment, the storage 34, for example, stores staff information 341, definition information 342 and registration information 343 as illustrated in FIG. 2.

The staff information 341 is information about health professionals and contains names, staff IDs, belongings, and professions. For example, the staff information 341 is used to authenticate a health professional who accesses the hospital information system 1 and further to generate task/work information.

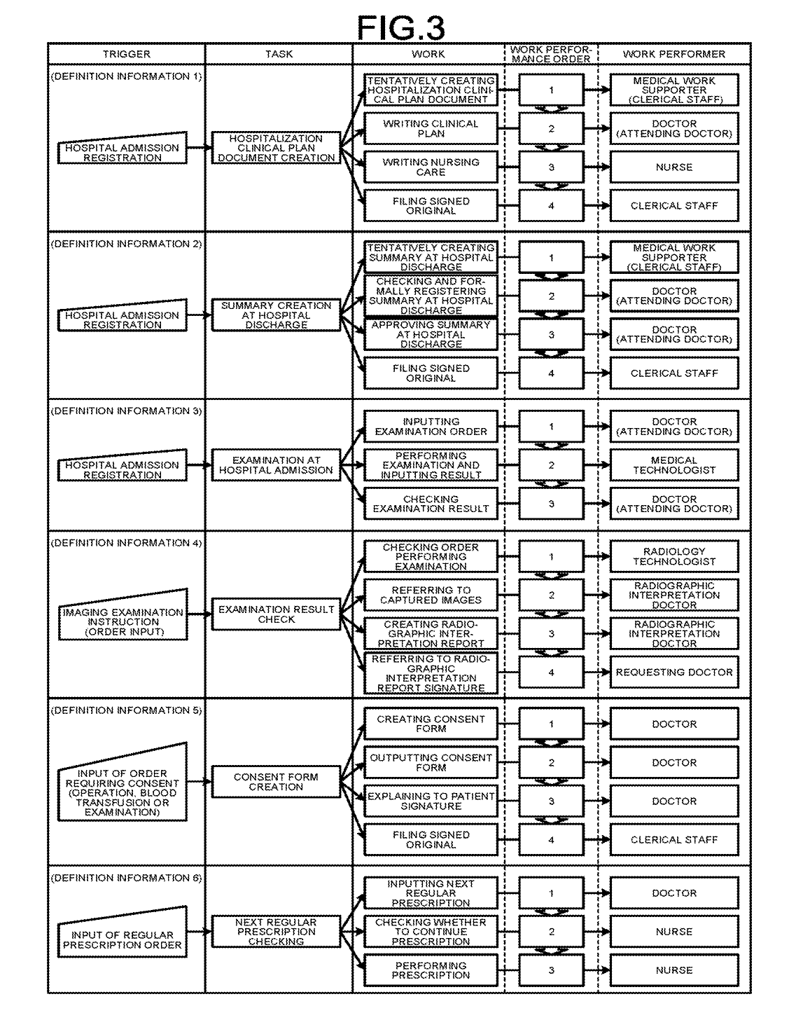

The definition information 342 is information that is used to generate task/work information and in which a task and works are associated with each other for each event serving as a trigger. It is possible to set the definition information 342 for each medical institution optionally and update the definition information 342 as appropriate. FIG. 3 is a diagram illustrating the exemplary definition information 342 according to the first embodiment. For example, the definition information 342 is information in which “Trigger”, “Task”, “Work”, “Work performance order”, and “Work performer” are associated with one another as illustrated in FIG. 3. A “trigger” in FIG. 3 represents an event that triggers generation of task/work information. A “task” represents a task that is performed in the medical institution. “Works” represent works that are performed to perform the “task”. A “work performance order” represents an order in which the “works” are performed. “Work performers” represent health professionals who perform the corresponding “works”.

For example, the storage 34 stores “definition information 1” in which “task: hospitalization clinical plan document creation” is associated with “trigger: hospital admission registration” and “work: tentatively creating hospitalization clinical plan document, work performance order: 1, work performer: a medical work supporter (clerical staff)”, “work: writing a clinical plan, work performance order: 2, work performer: a doctor (an attending doctor)”, “work: writing nursing care, work performance order: 3, work performer: a nurse”, and “work: filing the signed original, work performance order: 4, work performer: clerical staff” are associated with “task: hospitalization clinical plan document creation”.

The definition information 1 means that it is defined that performance of “hospital admission registration” triggers generation of task/work information in which a task of “hospitalization clinical plan document creation” and works corresponding to the task are associated with each other. The definition information 1 further means that it is defined that a task of “hospitalization clinical plan document creation” consists of a work “writing a hospitalization clinical plan” that is performed by a “clinical staff”, a work “tentatively writing a hospitalization clinical plan document” that is performed by a “clerical staff”, a work “writing a hospitalization clinical plan” that is performed by a “doctor (attending doctor)”, a work “writing nursing care” that is performed by a “nurse”, and a work “filing the singed original” that is performed by a “clerical staff” and that the works are performed in the following order: “creating a hospitalization clinical plan document”, “writing a clinical plan”, “writing nursing care” and “filing the signed original”.

The storage 34 further stores various types of definition information like “definition information 2” to “definition information 6” in FIG. 3. As for “definition information 2” and “definition information 3”, a task “summary creation at hospital discharge” and a task “examination at hospital admission” are associated with the same trigger “hospital admission registration” as that of “definition information 1”. In other words, according to the definition information 342 illustrated in FIG. 3, performance of “hospital admission registration” triggers generation of three sets of task/work information.

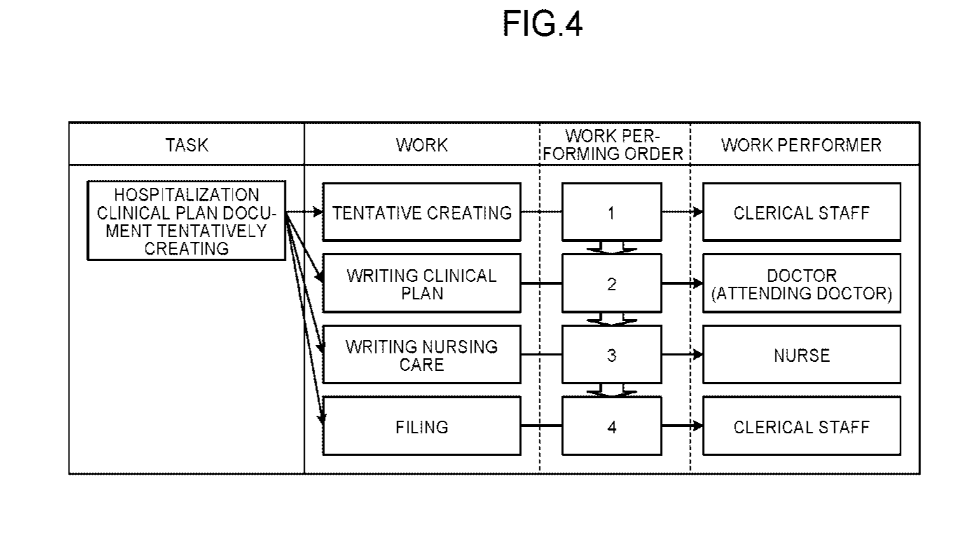

FIG. 2 will be referred back to. The registration information 343 is information that is stored by the processing circuitry 35, which will be described below, in the storage 34. Specifically, the registration information 343 is task/work information that is generated by the processing circuitry 35 in response to the trigger. FIG. 4 is a diagram illustrating the exemplary registration information 343 according to the first embodiment. For example, as illustrated in FIG. 4, the registration information 343 is information in which “task”, “work”, “work performance order” and “work performer” are associated with one another.

For example, the storage 34 stores registration information (task/work information) in which “work: tentatively creating, work performance order: 1, work performer: clerical staff”, “work: writing a clinical plan, work performance order: 2, work performer: doctor (attending doctor)”, “work: writing nursing care, work performance order: 3, work performer: nurse” and “work: filing, work performance order: 4, work performer: clerical staff” are associated with “task: hospitalization clinical plan document”.

FIG. 2 will be referred back to. The processing circuitry 35 executes a controlling function 351, a receiving function 352, a generating function 353, an updating function 354 and an outputting function 355. The processing circuitry 35 is an example of the processing circuitry according to the claims.

The controlling function 351 of the processing circuitry 35 controls the entire HIS server 3. For example, the controlling function 351 executes various processes according to input operations that are received from the operator via the input interface 32. For example, the controlling function 351 stores the staff information 341 that is input via the input interface 32 and the definition information 342 in the storage 34. The controlling function 351 implements control to display various types of information and a GUI on the display 33.

The receiving function 352 receives information that is received via the communication interface 31. For example, the receiving function 352 receives information about an input operation serving as a trigger, an input operation for performing a work, and an input operation for browsing task/work information from the terminal device 2 via the communication interface 31. For example, the receiving function 352 receives information representing that hospital admission registration of a subject is performed.

The generating function 353 generates the registration information 343 and stores the registration information 343 in the storage 34. Specifically, the generating function 353 is triggered, by determination of an event about medicine to the subject, to specify a task that relates to the event and a work of each health professional that is performed before completion of the task. The generating function 353 generates task/work information in which the specified task and works are associated with each other and stores the task/work information in the storage 34. The generating function 353 further specifies an order in which the works of the respective health professionals are performed and generates task/work information containing the specified order.

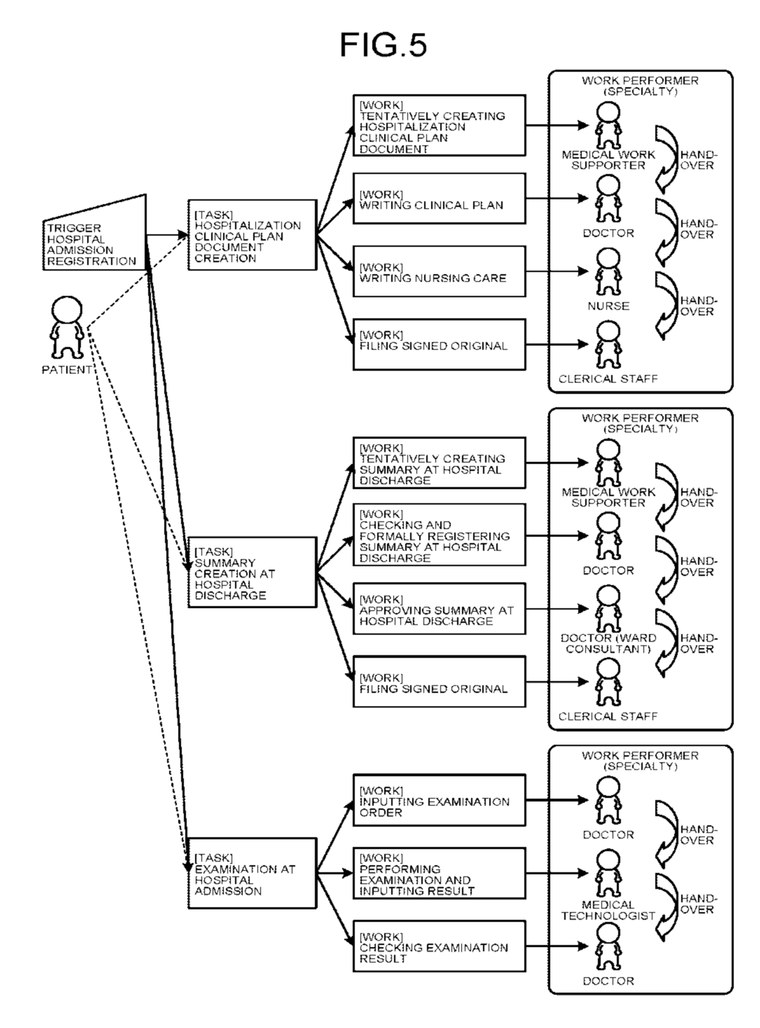

Specifically, the generating function 353 reads definition information corresponding to information about the trigger that is received by the receiving function 352 from the storage 34 and generates task/work information. FIG. 5 is a diagram for explaining an exemplary task/work information generation process that is performed by the generating function 353 according to the first embodiment. As illustrated in FIG. 5, when “hospital admission registration” of a subject (patient) is performed on the terminal device 2, the receiving function 352 receives the performed “hospital admission registration” as the information about the trigger.

The generating function 353 refers to the definition information 342 and reads definition information in which “hospital admission registration” that is received by the receiving function 352 serves as a trigger. For example, the generating function 353 reads “definition information 1”, “definition information 2” and “definition information 3” in which “hospital admission registration” serves as a trigger from the definition information 342 illustrated in FIG. 3 and generates task/work information on the basis of the sets of definition information that are read. For example, as illustrated in FIG. 5, the generating function 353 generates task/work information in which “work: tentatively creating a hospitalization clinical plan document”, “work: writing a clinical plan”, “work: writing nursing care” and “work: filing the signed original” are associated with “task: hospitalization clinical plan document creation”. The generating function 353 refers to the work performance order that is contained in “the definition information 1” and specifies an order in which the work performers perform the respective works. In other words, the generating function 353 refers to “the definition information 1”, specifies that “a medical work supporter”, “a doctor”, “a nurse” and “a clerical staff” succeed the work in the order they appear in this sentence and generates task/work information representing the specified performance order.

As described above, the generating function 353 refers to definition information corresponding to information about a trigger, generates task/work information and stores the task/work information in the storage 34. For example, the generating function 353 stores the task/work information illustrated in FIG. 4 as registration information in the storage 34. The generating function 353 refers to the information about the trigger and the staff information 341 and associates the information about the performers who perform the respective works with the task/work information. For example, works to complete “task: hospitalization clinical plan document creation” are performed by “a medical work supporter”, “a doctor”, “a nurse” and “a clerical staff”. The generating function 353 determines a performer of each work on the basis of information about “hospital admission registration” serving as a trigger and the staff information 341. For example, the generating function 353 specifies an attending doctor from the information about the doctor contained hospital admission registration and associates the information about the specified attending doctor with the task/work information. The generating function 353 further associates information about the clerical staff who performs hospital admission registration with the task work/information. The generating function 353 further specifies a nurse who makes plans nursing care on the basis of the staff information 341 and associates information about the specified nurse with the task/work information. For example, the generating function 353 stores the staff IDs of the performers as information about the performers of the respective works in association with the task/work information in the storage 34.

In the same manner, the generating function 353 generates task/work information in which “work: tentatively creating summary at hospital discharge”, “work: checking and finally registering a summary”, “work: approving the summary at hospital discharge” and “work: filing the signed original” are associated with “task: summary creation at hospital discharge” and stores the task/work information in the storage 34. Furthermore, as illustrated in FIG. 5, the generating function 353 generates task/work information in which “work: inputting an examination order”, “work: performing examination and inputting result” and “work: checking an examination result” are associated with “task: examination at hospital admission” and stores the task/work information in the storage 34.

FIG. 2 will be referred back to. The updating function 354 determines whether the work of each health professional that is specified by the generating function 353 is performed and updates registration information on the basis of the result of the determination. Specifically, the updating function 354 adds status information to the task/work information that is generated by the generating function 353. The updating function 354 determines whether each work is performed on the basis of the input operation for performing a work that is received by the receiving function 352 and updates the status in the task/work information. For example, when the receiving function 352 receives information representing that the document is registered (saved) in the system or that a document in a given format is printed out, the updating function 354 determines that the work is performed and then updates the status in the task/work information. The updating function 354 updates the status of the works on the basis of the results of the determination on whether the work of each health professional is performed and updates the status of the task on the basis of the status of the works.

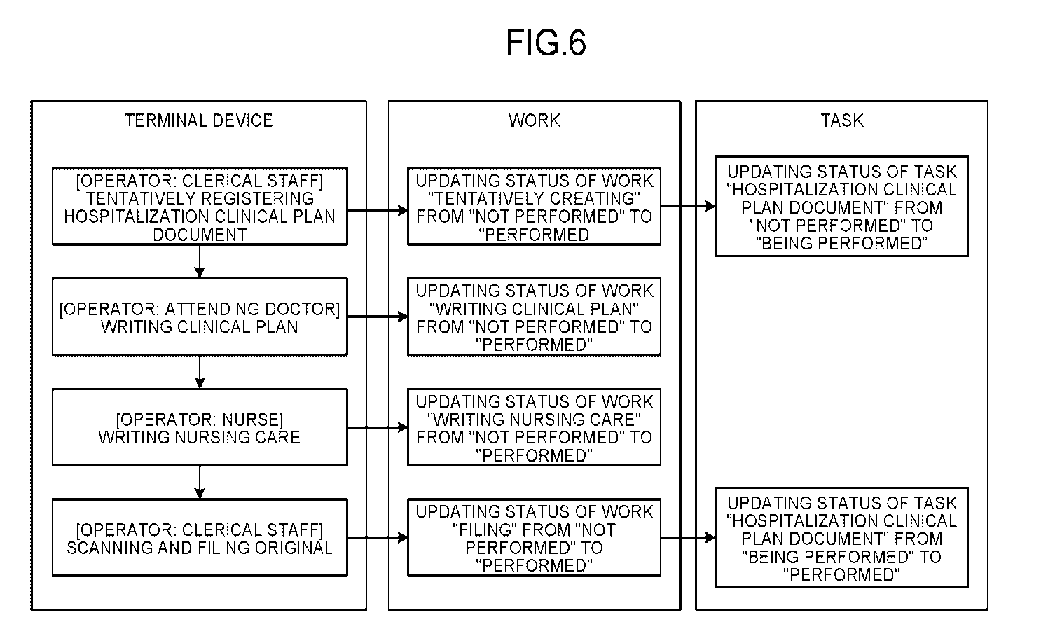

FIG. 6 is a diagram for explaining an exemplary updating process that is performed by the updating function 354 according to the first embodiment. FIG. 6 illustrates the process to perform the task of “hospitalization clinical plan document creation”. For example, as illustrated in FIG. 6, the updating function 354 determines that a clerical staff operates the terminal device 2 and tentatively registers a “hospitalization clinical plan document” and the updating function 354 updates the status of the work “tentative creation” from “not performed” to “performed” in the task/work information. Furthermore, as the status of the work “tentative creation” turns to be “performed”, the updating function 354 updates the status of the task “hospitalization clinical plan document” from “not performed” to “performed”.

The updating function 354 then determines that the attending doctor operates the terminal device 2 to write “a clinical plan” and to register the clinical plan in the system and the updating function 354 updates the status of “clinical plan writing” from “not performed” to “performed”. The updating function 354 then determines that a nurse operates the terminal device 2 to write “nursing care” and register “the nursing care” in the system and the updating function 354 updates the status of the work “nursing care writing” in the task/work information from “not performed” to “performed”. Furthermore, the updating function 354 determines that a clerical staff operates the terminal 2 to “scan and file the original” and the updating function 354 updates the status of the work “filing” in the task/work information from “not performed” to “performed”.

After updating all the works contained in the task to “performed” as described above, the updating function 354 updates the status of the task “hospitalization clinical plan document” from “being performed” to “performed”. Whether a document is registered in the system is, for example, determined according to whether the document is stored in a given folder in the system. Furthermore, whether the original is scanned and filed is determined according to whether a PDF file that is obtained by scanning the original is stored in a given folder.

FIG. 2 will be referred back to. The outputting function 355 implements control to output registration information representing the work of each health professional that is specified by the generating function 353 to the health professional. Specifically, when the receiving function 352 receives information corresponding to an input operation for browsing the task/work information from the terminal device 2, the outputting function 355 reads the task/work information corresponding to the information received by the receiving function 352 from the storage 34 and causes the read task/work information to be output from the display 23 of the terminal device 2. In other words, the outputting function 355 outputs the task/work information via the communication interface 21 to the terminal device 2 and accordingly the processing circuitry 25 of the terminal device 2 causes the display 23 to display the task/work information.

For example, when the receiving function 352 receives a request to browse the task/work information about a subject, the outputting function 355 reads the task/work information about the subject corresponding to the browsing request, which is received by the receiving function 352, from the storage 34 and outputs the task/work information to the terminal device 2 via the communication interface 21. Furthermore, for example, when the receiving function 352 receives a request to browse task/work information about a given health professional, the outputting function 355 reads task/work information about the health professional corresponding to the browsing request, which is received by the receiving function 352, from the storage 34 and outputs the task/work information to the terminal device 2 via the communication interface 21.

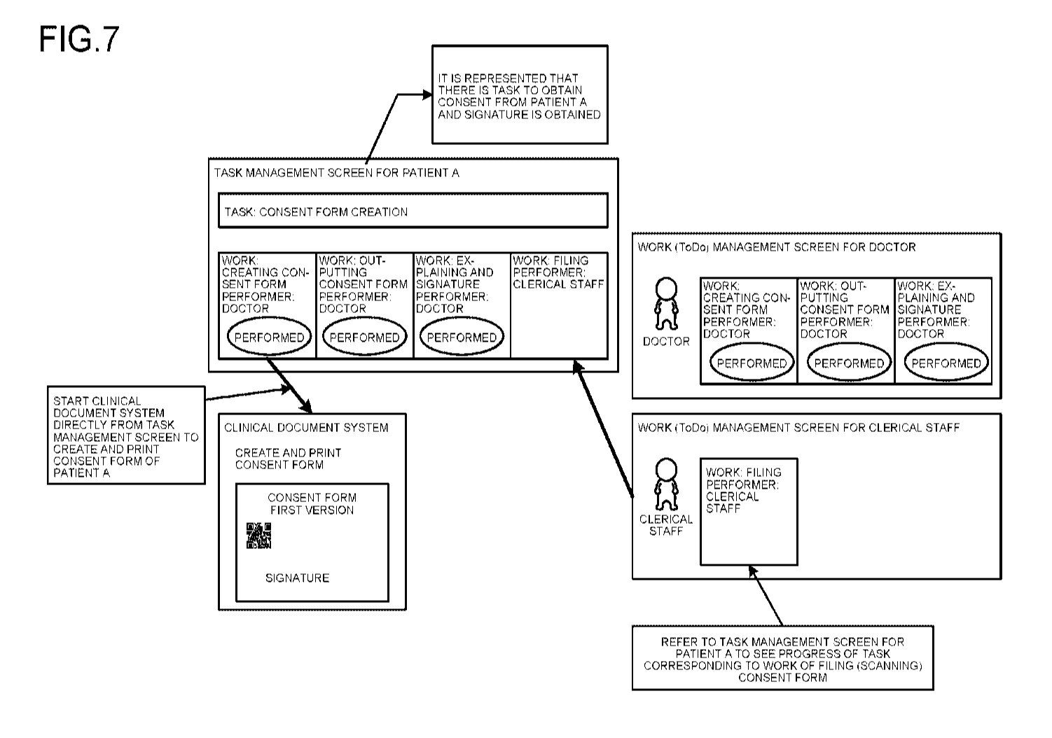

FIG. 7 is a diagram illustrating an overview of output of task/work information that is performed by the outputting function 355 according to the first embodiment. FIG. 7 illustrates exemplary output of task/work information about a subject (a patient A), exemplary output of task/work information about a doctor, and exemplary output of task/work information about a clerical staff. For example, as illustrated in FIG. 7, the outputting function 355 outputs a task management screen for the patient A as task/work information about the subject A to the terminal device 2. The outputting function 355 outputs, to the terminal device 2, a task management screen in which “a task: consent form creation” and four works “creating consent form”, “outputting the consent form”, “explanation and signature” and “filing” are associated with information about the performers and status of each of the works.

As for the task management screen that is output by the outputting function 355, a screen for performing a work from each work area may be directly started. For example, the operator clicks an area of “work: consent form creation”, so that the clinical document system is started and accordingly a consent form creation screen to obtain a signature from the patient A can be started. The operator is able to create and print a consent form by using the started clinical document system. As for the task management screen that is output by the outputting function 355, status information is represented for each work and, as “work: explanation and signature” is “performed”, the operator is able to know that a sign has been obtained from the patient A.

Furthermore, as illustrated in FIG. 7, the outputting function 355, for example, outputs a work (ToDo) management screen for doctor and a work (ToDo) management screen for clerical staff as the task/work information about the doctor and the task/work information about the clerical staff to the terminal device 2. For example, the outputting function 355 display “work: creating a consent form”, “work: outputting the consent form” and “work: signature” on the work (ToDo) management screen for the attending doctor of the patient A. The outputting function 355 further displays “work: filing” on the work (ToDo) management screen for the responsible clerical staff.

The task management screen for the subject (the patient A) and the work (ToDo) management screens for the health professionals (the attending doctor of the patient A and the responsible clerical staff) are linked with each other and the health professionals who refer to the screens can switch the screen. For example, a clerical staff who is referring to the work (ToDo) management screen for the clerical staff is able to switch the work (ToDo) management screen for the clerical staff to the task management screen for the patient A in order to check the progress of the task containing “work: filing”.

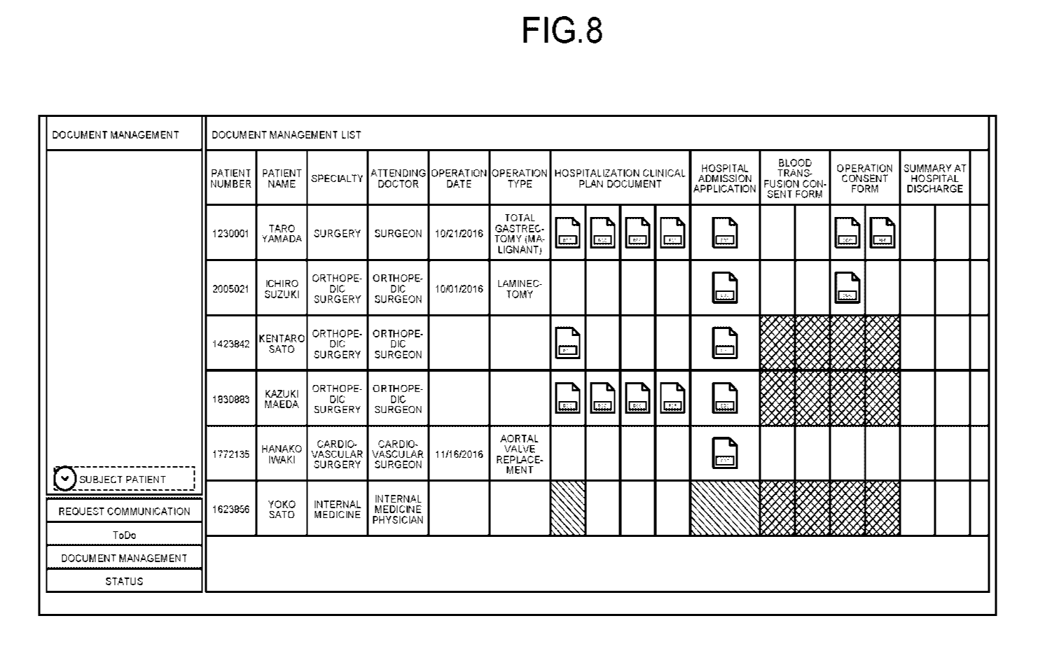

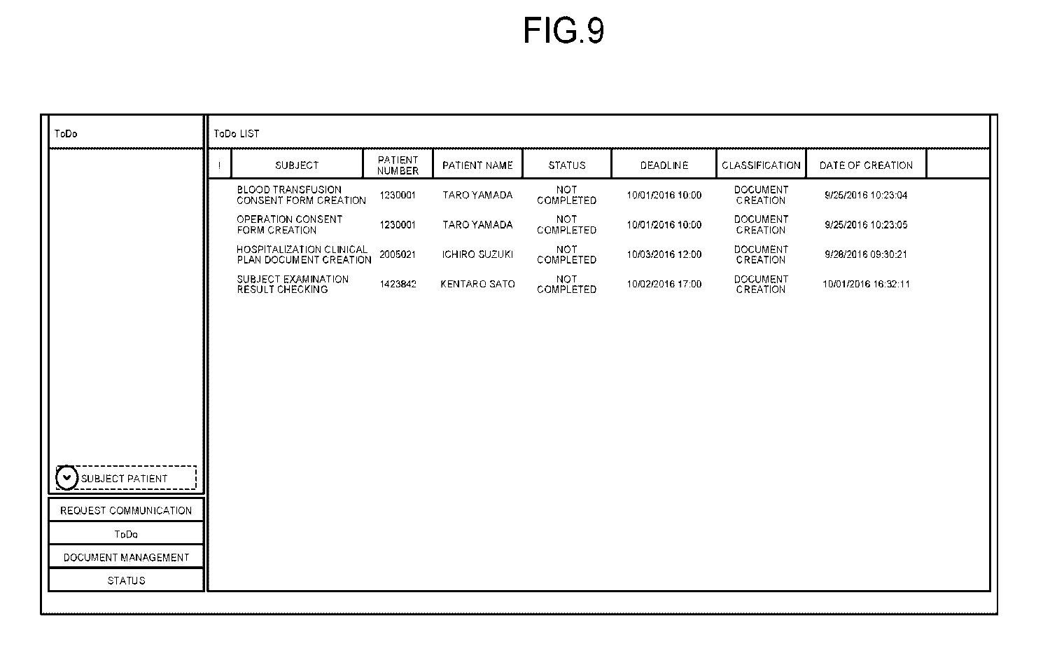

The overview of the display information (task/work information) that is displayed on the display 23 of the terminal device 2 under the control of the outputting function 355 has been described. Exemplary display information that is displayed on the display 23 of the terminal device 2 under the control of the outputting function 355 will be described with reference to FIGS. 8 and 9. FIGS. 8 and 9 are diagrams representing exemplary display information that is output by the outputting function 355 according to the first embodiment. FIG. 8 represents display information representing management information according to each subject. FIG. 9 represents display information representing management information about according to each health professional.

For example, as illustrated in FIG. 8, the outputting function 355 outputs display information that manages documents for each subject (patient). For example, the outputting function 355 outputs a document management screen in which “specialty: surgery”, “attending doctor: surgeon”, “operation date: 10/21/2016”, and “operation type: total gastrectomy (malignant)” are associated and task/work information about the related documents is associated with “patient name: Taro Yamada” corresponding to “patient number: 1230001”.

As illustrated in FIG. 8, the outputting function 355 outputs task/work information in which each task is represented by a document name and works are represented in the columns of display areas representing document icons. For example, the outputting function 355 displays a management screen in which four columns of icon display areas are provided to the item of “hospitalization clinical plan document”. This represents that “task: hospitalization clinical plan document” consists of four works. In other words, the four columns of icon display areas correspond to “work: tentatively creating”, “work: writing a clinical plan”, “work: wringing nursing care” and “work: filing” of “task: hospitalization clinical plan document”, respectively.

For example, when a responsible clerical staff tentatively creates a hospitalization clinical plan document for “patient number: 123001, patient name: Taro Yamada” and stores the hospitalization clinical plan in a given folder in the system, the updating function 354 updates the status of “work: tentatively creating” to “performed”. As the status in the registration information is “performed”, the outputting function 355 displays a document (DOC) icon in an icon display area on the left end of the item of “hospitalization clinical plan document” on the management screen.

The attending doctor (surgeon) of “patient number: 123001, patient name: Taro Yamada” then writes a clinical plan on the tentatively created “hospitalization clinical plan document” and stores the “hospitalization clinical plan document” in a given folder in the system, the updating function 354 updates the status of “work: clinical plan writing” in the registration information to “performed”. As the status in the registration information is “performed”, the outputting function 355 displays a document (DOC) icon in the second icon display area from the left of the item “hospitalization clinical plan document” on the management screen. In the same manner, when a responsible nurse writes nursing care on the “hospitalization clinical plan document” and then stores the “hospitalization clinical plan” in a given folder in the system, the outputting function 355 displays a document (DOC) icon on the third icon display area from the left of the item of “hospitalization clinical plan document” on the management screen. When the responsible clerical staff creates a PDF file by scanning and loading the “hospitalization clinical plan document” signed by “patient number: 1230001, patient name: Taro Yamada” and stores the PDF file in a given folder in the system, the outputting function 355 displays a PDF icon on the fourth icon area from the item of “hospitalization clinical plan document” on the management screen.

As described above, the outputting function 355 represents the task/work information in which the tasks are represented by the document names and the works are represented in the icon display areas associated with the document names. This enables the operator to know the progress of each task only by checking whether an icon is displayed in each of the icon display areas. The outputting function 355 may output task/work information in which work names and performer names are written in the respective icon display areas to specify the order in which the works are performed.

For example, the operator refers to the item of “hospitalization clinical plan document” for “patient number: 2005021, patient name: Ichiro Suzuki” and, as no icon is displayed in any of the icon display areas, the operator is able to know that all the works are not performed yet. Thereafter, the operator is able to know the status of the works on the basis of the icon display in “hospitalization clinical plan document” of “patient number: 2005021, patient name: Ichiro Suzuki”.

The outputting function 355 is also able to represent that there is no corresponding work by representing icon display areas for each of which no task occurs in a different color as represented in the icon display areas of “blood transfusion consent form” and “operation consent form” of “patient number: 1423842, patient name: Kentaro Sato” in FIG. 8. Furthermore, the outputting function 355 is able to make an alert by representing a work whose deadline is approaching or missed in a color, such as red, as represented in the icon display area at the left end of “hospitalization clinical plan document” of “patient number: 1623856, patient name: Yoko Sato”.

Each of the icon display areas may be linked with the clinical document system and document data that is stored in a given folder. In other words, by operating an icon display area, the operator is able to start the clinical document system for creating a corresponding document or read a document stored in a given folder and display the document. For example, by clicking the icon display area on the left end of the “hospitalization clinical plan document” of “patient number: 2005021, patient name: Ichiro Suzuki”, the operator is able to start the clinical document system for creating a “hospitalization clinical plan document” to create a “hospitalization clinical plan document”. Furthermore, by clicking the DOC icon that is represented in the icon display area at the left end of the “hospitalization clinical plan document” of “patient number: 1423842, patient name: Kentaro Sato”, the operator is able to start the clinical document system and read the “hospitalization clinical plan document” that is stored in a given folder in the system.

As illustrated in FIG. 9, the outputting function 355 further outputs display information to manage the documents for each health professional. For example, the outputting function 355 outputs a ToDo list screen in which “subject name”, “patient number”, “patient name”, “status”, “deadline”, “classification” and “date of creation” are associated with one another. The ToDo list screen represented in FIG. 9 represents works that are classified for each health professional who is identified by a staff ID. In other words, when the receiving function 352 receives a trigger and the generating function 353 registers registration information, the outputting function 355 classifies the works for each staff ID on the basis of the staff IDs that are associated with the works in the registration information and outputs the classified works to the ToDo list screen for each of the health professionals.

For example, the outputting function 355 displays, on the ToDo list screen, works corresponding to the staff ID that is authenticated to access the hospital information system 1 from among the works that are classified according to the staff IDs. The outputting function 355 is able output a work to the ToDo list screen at some sets of timing. For example, the outputting function 355 is able to output information about works to the ToDo list screen with respect to all the health professionals who perform the works contained in the task at the timing when the generating function 353 registers the registration information.

Alternatively, the outputting function 355 is able to output information about a work to the ToDo list screen at the timing when the turn of each health professional to perform the work comes. For example, after a clerical staff tentatively creates a “hospitalization clinical plan document”, a work may be output to the ToDo list screen for a doctor who writes a clinical plan on the “hospitalization clinical plan document”. In that case, the outputting function 355 determines whether the “hospitalization clinical plan document” that is tentatively created by the clerical staff is stored in a given folder and, at the timing when the “hospitalization clinical plan document” is stored in the given folder, outputs a work to the ToDo list screen for the doctor. In other words, the outputting function 355 refers to the order in which the works are performed, which is the order associated with the registration information, and performance of a work to be performed in an early turn triggers the outputting function 355 to output information about the next work to the ToDo list screen for the performer of the next work. The above-described output of works to the ToDo list screen is performed when the order in which the works are performed is clear. For example, when there is no clear order in which works are performed with respect to, for example, “work: writing a clinical plan” and “work: writing nursing care” of “task: hospitalization clinical plan document”, the outputting function 355 outputs information about works to the ToDo list screen for all the health professionals who perform the works contained in the task at the timing when the generating function 353 registers registration information.

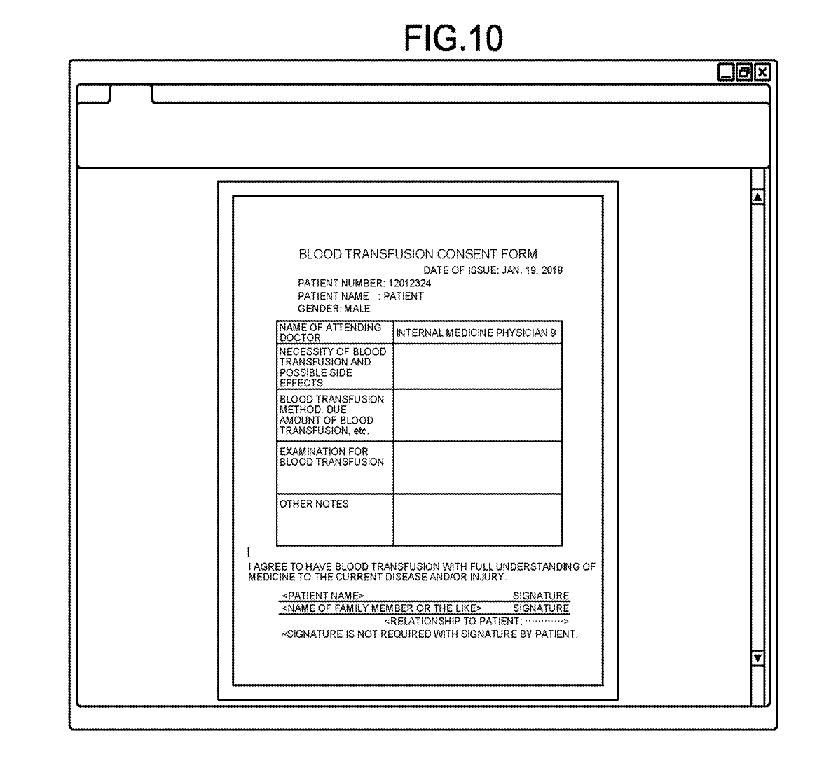

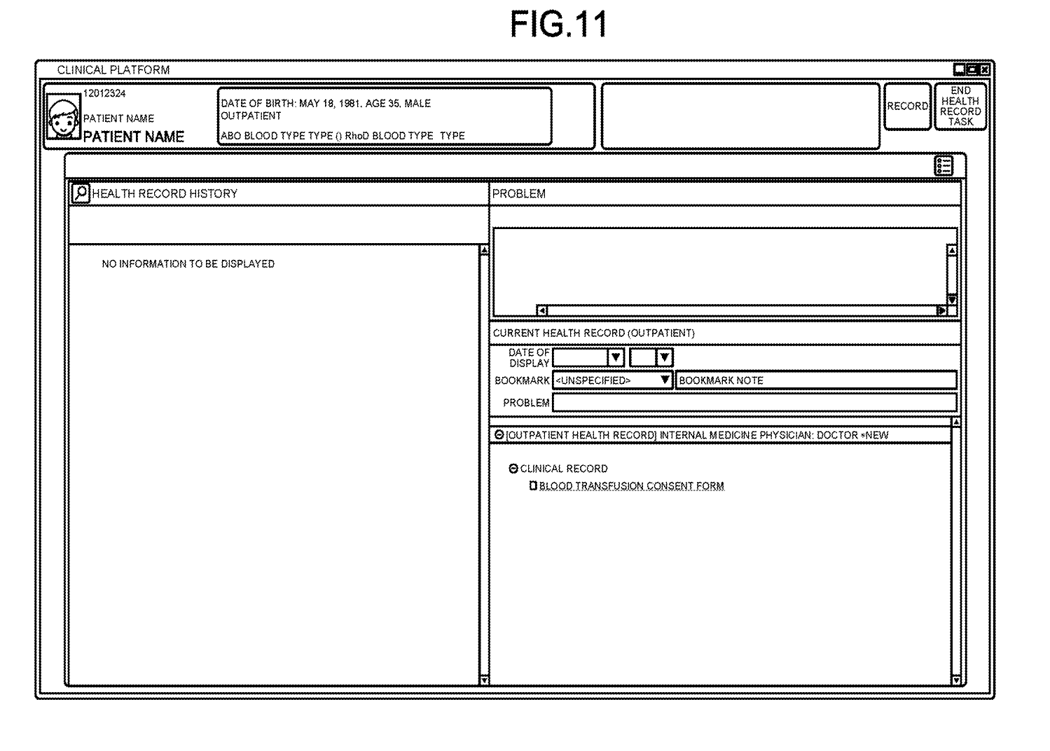

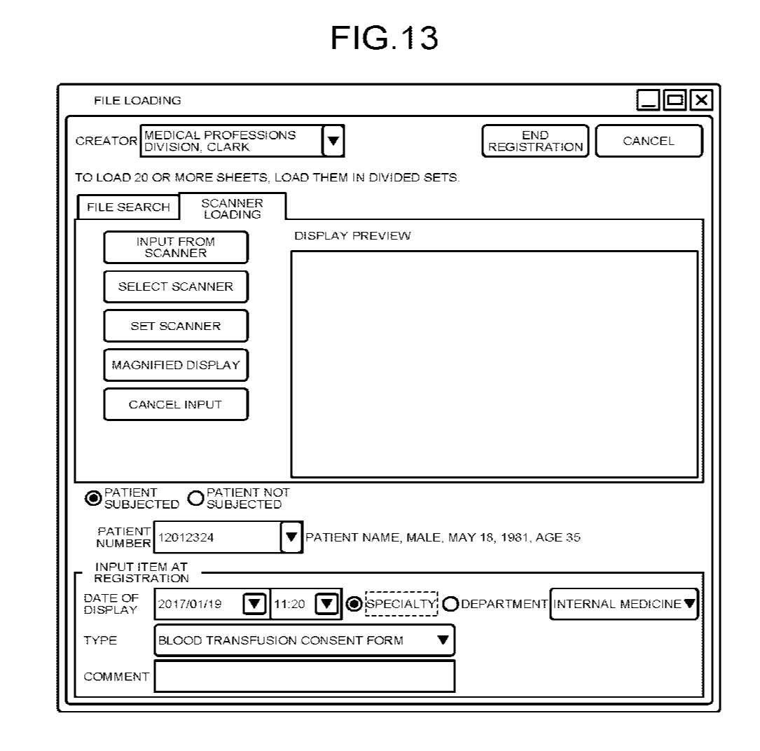

Exemplary display information that is displayed on the display 23 of the terminal device 2 under the control of the outputting function 355 has been described. Details of a process of determining whether each work is performed will be described with reference to FIGS. 10 to 14. FIGS. 10 to 14 are diagrams for explaining the determination process performed by the updating function 354 according to the first embodiment. Performance of each work of “task: blood transfusion consent form creation” will be exemplified and described below. FIG. 10 represents an exemplary blood transfusion consent form. FIGS. 11, 12 and 14 represent an exemplary electronic health record of a subject. FIG. 13 illustrates an exemplary scanning screen.

For example, a doctor who creates a blood transfusion consent form refers to the screen to manage documents for each subject, which is represented in FIG. 8, and clicks the left one of the two icon display areas associated with the item blood transfusion consent form of a subject for whom a consent form is currently created, thereby starting a document file of the blood transfusion consent form illustrated in FIG. 10. The doctor then makes an input to each item in the started document file. The doctor then registers the created blood transfusion consent form via the electronic health record of the subject for which the consent form is currently created.

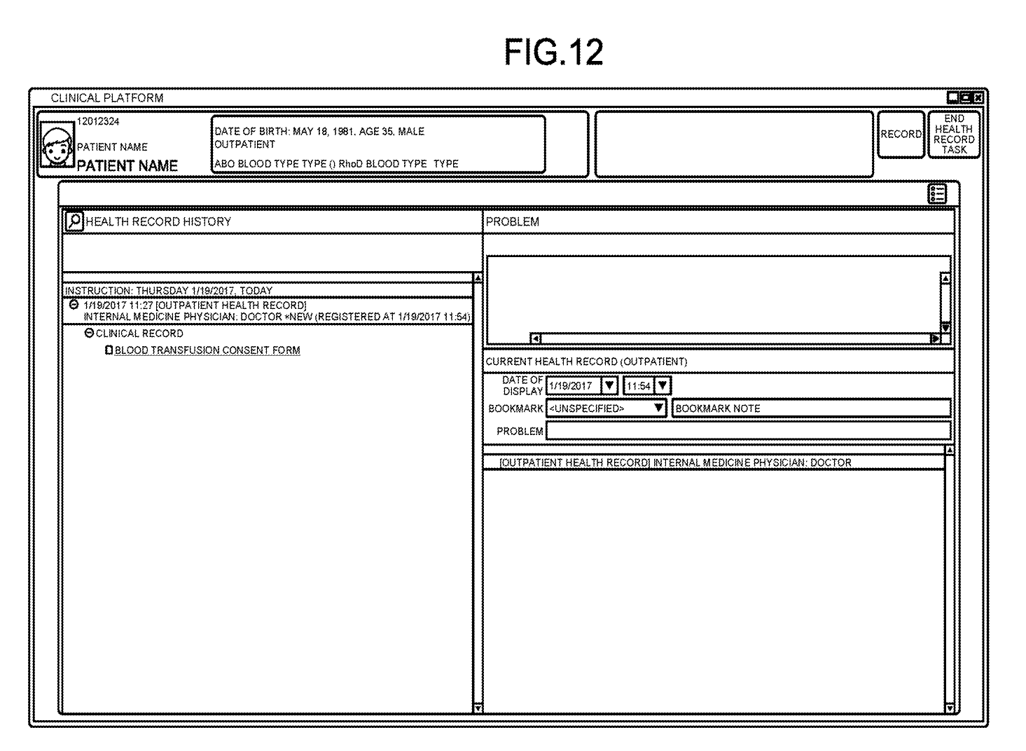

For example, starting the document file from the item of blood transfusion consent form of the subject on the document management screen enables, as illustrated in FIG. 11, loading of “blood transfusion consent form” in the clinical record of “outpatient health record” in “current health record (outpatient)” in the electronic health record of the subject for which a consent form is to be created. When the doctor who creates the blood transfusion consent form obtains a signature on the created blood transfusion consent form from the subject, the doctor executes a determination operation for registering the file of the blood transfusion consent form that is loaded into the electronic health record (clicking the record button in FIG. 11), thereby storing the file of the blood transfusion consent form in a given folder in the system. For example, when the doctor executes the determination operation, as illustrated in FIG. 12, “blood transfusion consent form” is loaded into the clinical record in “health record history” of the electronic health record of the subject.

For example, according to whether the doctor executes the determination operation to store the blood transfusion consent form is stored in the given folder in the system, the updating function 354 determines whether the work of blood transfusion consent form is performed. In other words, the updating function 354 determines whether the work of blood transfusion consent form is performed according to whether a DOC file of the blood transfusion consent form is registered in the health record history of the subject. The updating function 354 updates the status in the registration information. The original blood transfusion consent form signed by the subject is passed to the responsible clerical staff.

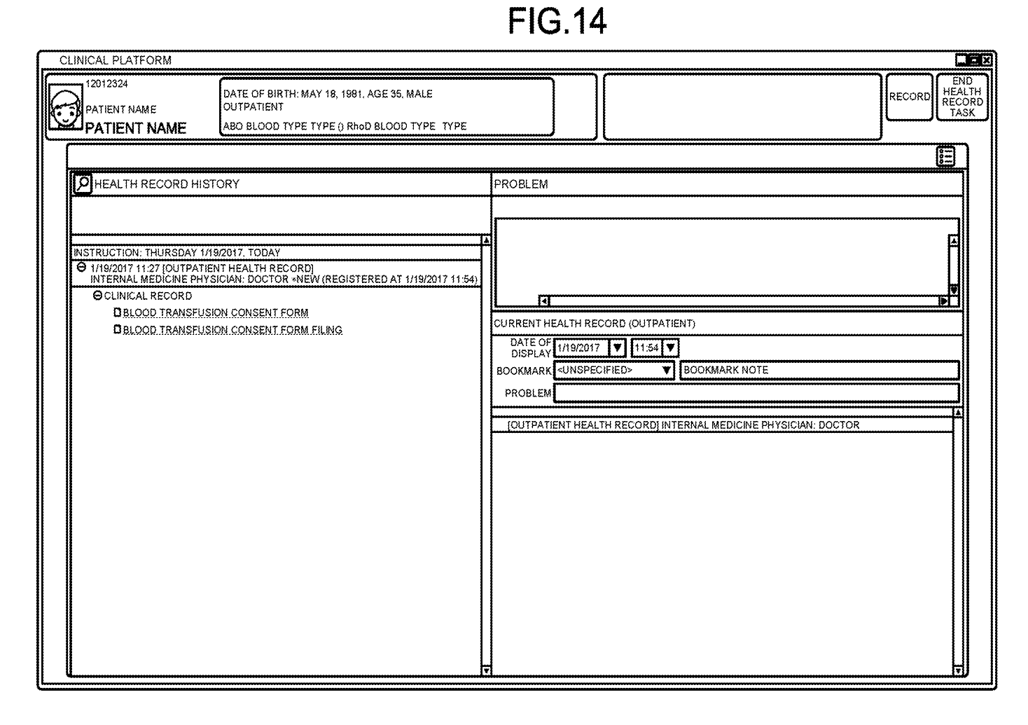

As described above, when the doctor creates and registers a blood transfusion consent form and the status in the registration information is updated, the outputting function 355 displays an icon of DOC file in a corresponding icon display area on the screen to manage documents for each subject, which is represented in FIG. 8. A clerical staff who scans and files the original blood transfusion consent form operates the terminal device 2 to start the scanning screen illustrated in FIG. 13. When the clerical staff scans the original blood transfusion consent form by a scanner, a preview of the scanned blood transfusion consent form is displayed in the scanning screen illustrated in FIG. 13. The clerical staff checks the scanning result by the preview. When the clerical staff executes the determination operation, a PDF file of the scanned blood transfusion consent form is stored in a folder in the system and, as illustrated in FIG. 14, “blood transfusion consent form filing” is loaded into the clinical record in “health record history” of the electronic health record of the subject.

For example, the clerical staff executes the determination operation and, according to whether a PDF file of the blood transfusion consent form is stored in a folder in the system, the updating function 354 determines whether the work of blood transfusion consent form is performed. In other words, the updating function 354 determines whether the work of filing is performed according to whether a PDF file of the blood transfusion consent form is registered in the health record history of the subject. The updating function 354 then updates the status of the registration information.

As described above, the updating function 354 determines whether a work is performed according to whether a file is registered in the system. The above-described example is an example only and the embodiments are not limited thereto. In other words, determination on whether the work is performed may be executed by using other information.

Each processing function of the processing circuitry 35 has been described. For example, each of the above-described processing functions is stored in a form of a computer-executable program in the storage 34. The processing circuitry 35 reads each of the programs from the storage 34 and, by executing each program that is read, implements the processing function corresponding to each of the programs. In other words, the processing circuitry 35 having read each of the programs has each of the processing functions represented in FIG. 2.

FIG. 2 illustrates the exemplary case where each of the processing functions is implemented by the single processing circuitry 35; however, the embodiments are not limited thereto. For example, the processing circuitry 35 may consist of a combination of multiple independent processors and the processors execute the respective programs and accordingly implement the respective processing functions. Alternatively, the processing functions of the processing circuitry 35 may be implemented in a manner that the processing functions are distributed to multiple processing circuits or integrated into a single processing circuit.

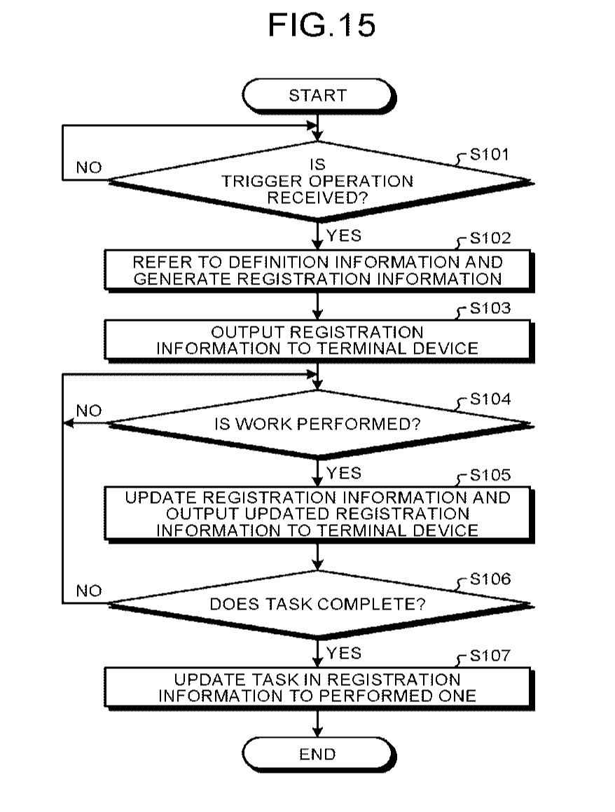

A process procedure performed by the hospital information system 1 according to the first embodiment will be described with reference to FIG. 15. FIG. 15 is a flowchart illustrating the process procedure performed by the hospital information system 1 according to the first embodiment. Step S101 in FIG. 15 is implemented, for example, by the processing circuitry 35 by reading the program corresponding to the receiving function 352 from the storage 34 and executing the program. Step S102 is implemented, for example, by the processing circuitry 35 by reading the program corresponding to the generating function 353 from the storage 34 and executing the program. Steps S103 and S105 are realized, for example, by the processing circuitry 35 by reading the program corresponding to the outputting function 355 from the storage 34 and executing the program. Steps S104, S106 and S107 are implemented, for example, by the processing circuitry 35 by reading the program corresponding to the updating function 354 from the storage 34 and executing the program.

For example, as illustrated in FIG. 15, in the hospital information system 1 according to the embodiment, first of all, the processing circuitry 35 determines whether a trigger operation is received (step S101). When it is determined that a trigger operation is received (YES at step S101), the processing circuitry 35 refers to the definition information 342 and generates the registration information 343 (step S102). The processing circuitry 35 then outputs the registration information 343 to the terminal device 2 (step S103). Note that the hospital information system 1 is in a standby state until a trigger operation is received (NO at step S101).

The processing circuitry 35 then determines whether a work is performed (step S104). When it is determined that a work is performed (YES at step S104), the processing circuitry 35 updates the registration information and outputs the updated registration information to the terminal device 2 (step S105). The processing circuitry 35 further determines whether a task completes (step S106). When it is determined that the task completes (YES at step S106), the processing circuitry 35 updates the task of registration information to the performed one (step S107).

As described above, according to the first embodiment, the generating function 353 is triggered, by determination of an event that relates to medicine to a subject, to specify a task that relates to the event and a work of each health professional to be performed before the task completes. The outputting function 355 implements control to output display information representing the work of each health professional, which is specified by the generating function 353, to the health professional. The hospital information system 1 according to the first embodiment is able to represent information about the work to each of the health professionals and thus enables each work to be performed speedily. As a result, the hospital information system 1 is able to improve efficiency of performing a task consisting of multiple works.

Furthermore, according to the first embodiment, the generating function 353 further specifies the order in which the works of the health professionals are performed. The outputting function 355 implements control to output display information representing the order in which the works of the health professionals are performed. Accordingly, the hospital information system 1 according to the first embodiment enables health professionals to know the progress of each task and perform the works to be performed by the health professionals speedily. Even when performance of a task delays, which work delays is represented and accordingly the hospital information system 1 according to the first embodiment is able to prompt the responsible health professional to perform the delayed work and enables further improvement in medical task efficiency.

According to the first embodiment, the outputting function 355 implements control to output display information about each subject. The outputting function 355 implements control to output display information about each health professional. Accordingly, the hospital information system 1 according to the first embodiment is able to represent the status of a task and works by using comprehensive information and thus further improve medical task efficiency.

According to the first embodiment, the updating function 354 determines whether the work of each health professional that is specified by the generating function 353 is performed and updates the display information according to the result of the determination. The outputting function 355 implements control to output the display information that is updated by the updating function 354 to the health professionals. The hospital information system 1 according to the first embodiment enables health professionals to know the progress of the task.

According to the first embodiment, the updating function 354 updates the status of works on the basis of the result of determination on whether the work of each health professional is performed and updates the status of the task on the basis of the status of the works. The outputting function 355 implements control to output display information containing the status of the works and the status of the task.

Accordingly, the hospital information system according to the first embodiment enables health professionals to know the detailed progress of the task.

### Second Embodiment

The first embodiment has been described above. In addition to the above-described first embodiment, various different modes may be carried out.

The above-described embodiment illustrates the case where the HIS server 3 executes each process; however, the embodiments are not limited thereto. For example, the terminal device 2 may execute each process. For example, the processing circuitry 25 of the terminal device 2 may execute the above-described updating function.

Furthermore, the above-described embodiment describes the example where the work (ToDo) management screens with works classified for each health professional. The Work (ToDo) management screen is not limited to the above-described exemplary display (the screen illustrated in FIG. 9). The display mode may vary as appropriate. For example, works whose deadline is approaching may be displayed in a different color.

The above-described embodiment exemplifies display information represented with respect to each subject and display information represented with respect to each health professional. The hospital information system 1 according to the embodiment may display health professionals such that the health professionals are identifiable with respect to each work in the display information. For example, the outputting function 355 implements control to output display information representing the specified work of each health professional so that the health professionals are identifiable with respect to each work in the display information. An exemplary process performed by the outputting function 355 will be described with reference to FIG. 8.

For example, when displaying display information about each subject represented in FIG. 8, the outputting function 355 causes display information representing works of the health professionals such that the works are identifiable. For example, the outputting function 355 adds a color different from that of other works or a mark to a work that is performed by a given health professional from among the works contained in the display information represented in FIG. 8 and displays the display information. In that case, for example, the outputting function 355 specifies a work of the health professional who executes an input operation to browse the display information (task/work information) from among the works contained in the display information represented in FIG. 8 on the basis of the staff ID of the health professional, adds a color different from that of other works or a mark to the specified works and displays the display information. The display mode enabling the work to be identifiable is not limited to using the above-described color or work, and any other mode employing, for example, enhanced display may be used.

The above-described embodiment describes that a work is output to the ToDo list screen relating to each health professional at various types of timing. The hospital information system 1 according to the embodiment may separately display works listed according to an order in which the works are to be performed and works to be performed later. Specifically, the outputting function 355 separately displays display information in which works that are already performable are listed and display information in which works to be requested later are listed. In that case, the outputting function 355 determines the status of the task to which each work corresponds, determines whether each work is already performable, and classifies each work on the basis of the result of the determination. On the basis of the result of the classification, the outputting function 355 separately displays the display information in which works that are already performable are listed and display information in which works to be requested later are listed.

The word “processor” used in the descriptions of each of the embodiments given above refers to, for example, a central processing unit (CPU), a graphics processing unit (GPU) or a circuit, such as an application specific integrated circuit (ASIC) or a programmable logic device (for example, a simple programmable logic device (SPLD), a complex programmable logic device (CPLD) or a field programmable gate array (FPGA)). Instead of being saved in a storage circuit, a program may be directly incorporated in a circuit of a processor. In this case, the processor implements functions by reading and executing the program that is incorporated in the circuit. Each processor of the embodiment is not limited to the case where each processor is configured as a single circuit, and multiple independent circuits may be combined into a single processor to implement the functions.

The program executed by the processor is incorporated in a read only memory (ROM) or a storage unit in advance and provided. The program may be provided by being stored in a file in a format enabling installation in and execution by the devices in a computer-readable recording medium, such as a compact disk (CD)-ROM, a flexible disk (FD), a CD-R (recordable) or a digital versatile disk (DVD). The program may be stored in a computer that is connected to a network, such as the Internet, and may be provided or distributed by being downloaded via the network. For example, the program may consist of modules containing the respective functional units. In practical hardware, the CPU reads the program from the storage medium, such as a ROM, and executes the program and accordingly each module is loaded into a main storage device and generated in the main storage device.

According to at least one of the above-described embodiments, it is possible to increase the medical task efficiency.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A hospital information system comprising:
processing circuitry configured to
specify a work of each health professional by using determination of an event that relates to medical care to a subject as a trigger, the work relating to the event, and
output display information representing the specified work of each health professional so that the health professionals are identifiable with respect to each work in the display information.

2. The hospital information system according to claim 1, wherein the processing circuitry is configured to
further specify an order in which the works of the respective health professionals are performed, and
output display information representing the order in which the works of the respective health professionals are performed.

3. The hospital information system according to claim 1, wherein the processing circuitry is configured to output the display information about each subject.

4. The hospital information system according to claim 1, wherein the processing circuitry is configured to output the display information about each health professional.

5. The hospital information system according to claim 1, wherein the processing circuitry is configured to
determine whether the specified work of each health professional is performed and update the display information on the basis of a result of the determination, and
output the updated display information.

6. The hospital information system according to claim 5, wherein the processing circuitry is configured to
update a status of the work of each health professional on the basis of a result of the determining whether the work of each health professional is performed and, on the basis of the status of the works, update a status of a task that is completed by performing the works of the respective health professionals, and
output display information containing the status of the works and the status of the task.

7. A hospital information system comprising:
processing circuitry configured to
specify a work of each health professional by using determination of an event that relates to medical care to a subject as a trigger, the work relating to the event, and
output display information representing the specified work of each health professional.

8. The hospital information system according to claim 7, wherein the processing circuitry is configured to
further specify an order in which the works of the respective health professionals are performed, and
output display information representing the order in which the works of the respective health professionals are performed.

9. The hospital information system according to claim 7, wherein the processing circuitry is configured to output the display information about each subject.

10. The hospital information system according to claim 7, wherein the processing circuitry is configured to output the display information about each health professional.

11. The hospital information system according to claim 7, wherein the processing circuitry is configured to
determine whether the specified work of each health professional is performed and update the display information on the basis of a result of the determination, and
output the updated display information.

12. The hospital information system according to claim 11, wherein the processing circuitry is configured to
update a status of the work of each health professional on the basis of a result of the determining whether the work of each health professional is performed and, on the basis of the status of the works, update a status of a task that is completed by performing the works of the respective health professionals, and
output display information containing the status of the works and the status of the task.
